(19) United States
(12) Patent Application Publication
(10) Pub. No.: US 2012/0021507 A1
(43) Pub. Date: Jan. 26, 2012

(54) BIOREACTOR AND USES THEREOF

(75) Inventor: Mizhou Hui, Thousand Oaks, CA (US)

(73) Assignees: AM-PROGRAM CORPORATION; ENGINEERING CO., LTD.; WASTEWATER TREATMENT; HANGZHOU JIUHUA QINGBO

(21) Appl. No.: 13/145,299

(22) PCT Filed: Jan. 20, 2010
(86) PCT No.: PCT/US10/21522

§ 371 Date: Sep. 30, 2011

Related U.S. Application Data
(60) Provisional application No. 61/145,893, filed on Jan. 20, 2009.
(60) Provisional application No. 61/205,590, filed on Jan. 21, 2009.
(60) Provisional application No. 61/212,387, filed on Apr. 11, 2009.
(60) Provisional application No. 61/168,740, filed on Apr. 13, 2009.
(60) Provisional application No. 61/258,322, filed on Nov. 5, 2009.

(51) Int. Cl. C02F 1/72; B01F 3/04; C02F 3/18; C12N 5/071; C12N 1/20; C12N 1/16; C12N 5/04; C12M 1/06; C12N 7/00

(52) U.S. Cl. 435/325; 435/289.1; 261/91; 210/150; 210/749; 210/758; 210/760; 210/629; 210/619; 435/235.1; 435/252.1; 435/255.1; 435/410

(57) ABSTRACT

Disclosed are bioreactor systems and methods to transfer a gaseous composition into a liquid composition in a closed environment. Also disclosed are bioreactor systems and methods for high-density culture of microorganisms and stably culture of activated sludge.

### RELATED APPLICATIONS

This application claims priority to U.S. provisional application No. 61/145,893, filed on Jan. 20, 2009; U.S. provisional application No. 61/168,740, filed on Apr. 13; 2009, U.S. provisional application No. 61/205,590, filed on Jan. 21, 2009; U.S. provisional application No. 61/212,387, filed on Apr. 11, 2009; and U.S. provisional application No. 61/258,322, filed on Nov. 5, 2009. The contents of these prior applications are herein incorporated by reference.

### FIELD OF DISCLOSURE

The disclosure relates to the methods and systems for introducing ambient gas into a liquid.

### BACKGROUND

There is a need to dissolve various gases in a liquid. For example, in waste-water treatment, it is useful to dissolve oxygen or ozone in water. One known waste-water treatment method includes introducing oxygen in the water to support growth of aerobic bacteria. This is typically achieved by bubbling oxygen through the water. It is also useful to add ozone to water for killing bacteria and viruses, as well as for removing odors and colors. Such treatments are used, for example, in processing fruits and vegetables. The introduction of ozone is again typically achieved by bubbling ozone in the water. A difficulty associated with conventional air-bubbling methods is their appetite for electricity. In addition, such methods are inefficient. When air-bubbling is used, a considerable amount of time elapses before the level of dissolved gas reaches a useful level. As a result, a great deal of gas fails to dissolve and is ultimately wasted.

### SUMMARY

The present invention relates to the use of a rotating wheel to introduce a gas into a liquid medium. The wheel is covered by one or more net structures. The structures are formed by having ribs interconnecting with each other to form voids. When the voids are regularly shaped, each layer of the structure can be viewed as a mesh layer, or a net layer.

The wheel can be mounted to protrude above the surface of a liquid. As the wheel rotates, the voids in the net structures trap air bubbles. As these bubbles interact with the boundaries between the voids (e.g. the ribs in adjacent layers of netting), they become progressively smaller, and are therefore more prone to dissolve in the liquid.

In one aspect, the invention features a rotating wheel assembly for introducing an ambient gas into a liquid. The assembly includes a wheel plate having a face; and a net structure on the face. The wheel plate can be solid or not permeable to a liquid, such as water. The rotating wheel assembly can further include an axle passing through the wheel plate, the axle being positioned such that a portion of the wheel plate protrudes above the level of the liquid. The net structure can be made of aluminum, aluminum alloy, stainless steel, or ozone-resistant plastic. It can contain one or more layers of plastic or metal net on the face. The net can have mesh having a shape of diamond, square, or hexagon. The mesh can be of 0.5-2.0 cm in diameter.

In one embodiment, the assembly has a wheelward-most layer from the layers of net that contains a plurality of meshes having a first size, and a wheelward-least layer from the plurality of net layers that contains a plurality of net meshs having a second size, wherein the second size is larger than the first size.

In another embodiment, the above-mentioned face is a side face or a circumferential face of the wheel plate. For example, the wheel is a rolling tube covered by one or more layers of the net structures. See FIG. 2.

The rotating wheel assembly can include a plurality of wheel plates, each having a face for placement of the net structure. It can include a first wheel plate; a second wheel plate mounted coaxially with the first wheel plate and separated from the first wheel plate along an axial direction; and a plurality of boards extending between the first and second wheels, the boards having faces. The net structure is disposed on the faces of the boards.

The invention also features a rotating wheel assembly for introducing a gas into a liquid. The assembly can include means for holding the liquid; means for entraining bubbles of the gas; and means for plunging the entraining means below a surface of the liquid and means for trapping and chopping off air bubbles of a large size into a smaller size.

In another aspect, the invention features an apparatus having multiple rotating wheel assemblies described above. The assemblies are assembled together in a bioreactor tank or chamber.

In another aspect, the invention features a bioreactor for introducing an ambient gas into a liquid. It contains a tank for holding the liquid; and a wheel assembly described above rotatably mounted in the tank. The bioreactor can further include an airtight lid for the tank. The gas can be air, oxygen, ozone, a fragrant gas, N₂, or CO₂. For air, oxygen, fragrant gas, N₂, or CO₂, the net structure can be made of plastic, aluminum, aluminum alloy, stainless steel, or plastic. For ozone, the net structure can be made of ozone-resistant plastic and the tank is airtight.

In the bioreactor, the liquid can contain chemicals, virus, microorganisms (e.g., bacteria or yeast), plant cells, or mammalian cells. The gas introduced into the liquid can treat the chemicals or kill the virus, microorganisms, plant cells, or mammalian cells. The liquid can contain water, industry wastewater, or sewage. Alternatively, the gas is required for the growth of the microorganisms or cells.

In one embodiment of the bioreactor, the tank further includes activated sludge, which can be used to treat polluted water. The activated sludge can be grown on a matrix, which can contain polymer non-woven cloth in between plastic nets. The activated sludge can be composed of saprotrophic bacteria but also can have a protozoan flora mainly composed of amoebae, Spirotrichs, Peritrichs including Vorticellids and a range of other filter feeding species. Other important constituents include motile and sedentary Rotifers.

In another aspect, the invention features a method of liquid treatment. The method includes repeatedly moving a net structure through a gas to be dissolved in the liquid; and plunging the net structure into the liquid, wherein the liquid is in a tank. The gas can be air, oxygen, ozone, fragrant gas, N₂, or CO₂. For air, oxygen, fragrant gas, N₂, or CO₂, the net structure can be made of plastic, aluminum, aluminum alloy, stainless steel, or plastic. For ozone, the net structure can be made of ozone-resistant plastic and the tank is airtight and the tank is airtight. The liquid can contain chemicals, virus, microorganisms, plant cells, or mammalian cells. The gas introduced into the liquid, can treat the chemicals or kill the virus, microorganisms (e.g., bacteria or yeast), plant cells, or mammalian cells. The liquid can contain water, industry wastewater, sewage, culture medium, or broth.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

FIG. 1 is a rotating wheel (made by organic glass or aluminum alloy or plastic) covered by multiple layers of nets for ambient gas transfer into water.

FIG. 2 is an illustration of detailed making of an exemplary ambient gas transfer rotating wheel.

FIG. 3 is an illustration of a novel oxygen transfer wheel method for generation of micro-bubbles and dissolved oxygen.

FIG. 4 is an ozone treatment rotating wheel bioreactor which is characterized by its capability to 100% transfer ambient ozone into water or wastewater in a sealed chamber.

FIG. 5 is an ozone treatment bioreactor which is characterized by its rotating ozone transfer wheel combined with an ozone transfer board to 100% transfer ambient ozone into water or wastewater in a sealed chamber.

FIG. 6 is a small-scale ozone treatment rotating wheel bioreactor for processing small-amount of wastewater.

FIG. 7 is a large-scale ozone treatment rotating wheel bioreactor for processing large-amount of wastewater.

FIG. 8 is ozone treatment rotating wheel bioreactor to clean or sterilize vegetables, seafood, meat, clothes and dishes.

FIG. 9 is a ground water cleaning or sterilizing ozone bioreactor system.

FIG. 10 is portable version of the treatment system of FIG. 5, a truck equipped with O₃ water treatment bioreactor.

FIG. 11 shows an experiment to compare oxygen transfer speeds of dissolved oxygen making wheel bioreactor and a conventional impellor/bubbling-based deep tank bioreactor. Air was used for the culture. Maximum speed of the impellor was 750 rpm while maximum rotating speed of the wheel was 190 rpm. A photo in panel 3 indicates that dissolved oxygen making wheel bioreactor generated significantly less shear force, namely much clear supernatant of the sample after centrifugation.

FIG. 12 is a photograph of rotating wheels (2.0 meter in diameter) under examination using our oxygen transfer wheel experimental platform.

FIG. 13 is a novel small-scale ozone treatment rotating wheel bioreactor system.

FIG. 14 is a novel large-scale ozone treatment rotating wheel bioreactor system.

FIG. 15 is a group of photos showing non-woven polymer cloth carrier packed in between two plastic nets for attached growth of activated sludge and microorganism.

FIG. 16 is a wastewater treatment bioreactor unit using rotating wheels of 0.5 meter in diameter for dissolved oxygen making.

FIG. 17 is a small-scale wastewater treatment bioreactor system using rotating wheels of 0.5 meter in diameter.

FIG. 18 is a medium-scale wastewater treatment bioreactor system using rotating wheels of 0.5, 1.0 and 2.0 meter in diameter for dissolving oxygen and ozone, and specially designed for making re-generated water.

FIG. 19 is a medium-scale wastewater treatment bioreactor system using rotating wheels of 0.5, 1.0 and 2.0 meter in diameter for dissolving oxygen and ozone, and specially designed for treatment of infectious wastewater from hospital and vaccine manufacturer.

FIG. 20 is a large-scale wastewater treatment bioreactor system using rotating wheels of 0.5, 1.0 and 2.0 meter in diameter for dissolving oxygen and ozone.

FIGS. 21A-F are a group of photos of a small-scale wheel bioreactor system (0.25 meter in diameter wheel). The complete system includes anaerobic fermentation, aerobic fermentation, O₃ treatment and Pi removal (a, b, c, d, e, and f). The system was used for small-scale pilot test of different kinds of wastewater.

FIG. 22 is a diagram showing a liquid treatment system with its treatment chamber in a closed position.

FIG. 23 is a diagram showing the liquid treatment chamber of FIG. 22 in its open position.

FIG. 24 is a rim view of the wheel shown in FIG. 23.

FIG. 25 is an exploded view of a stack of tessellation or net layers of a wheel similar to that shown in FIG. 24.

FIG. 26 is a diagram showing the system of FIG. 22 used for groundwater remediation.

FIG. 27 is a diagram showing a wheel having a wide rim with a net structure on the rim.

FIG. 28 is a diagram showing wheel assembly having co-axial wheels that collectively achieve the effect of the wide rim shown in FIG. 27.

FIG. 29 is a diagram showing a wheel assembly having boards extending axially between a pair of wheels.

### DETAILED DESCRIPTION

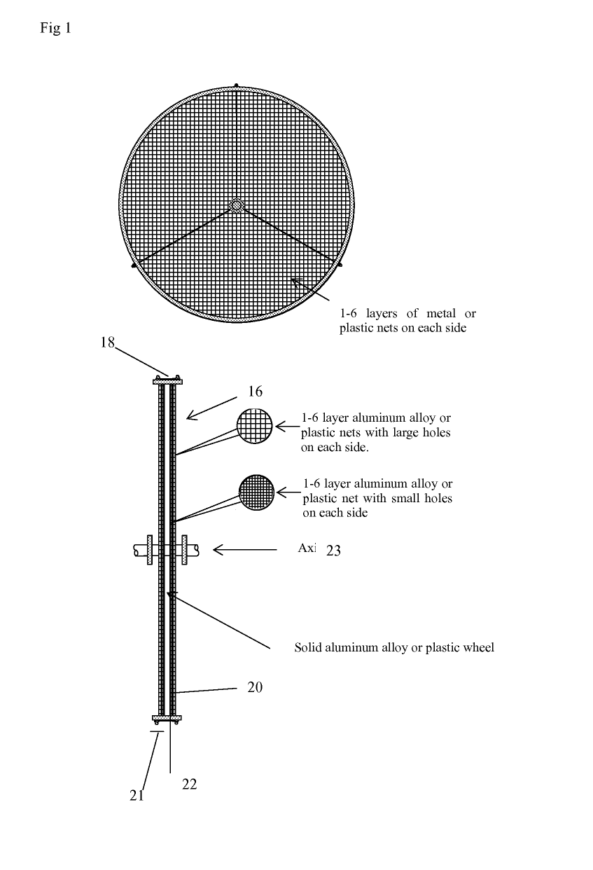

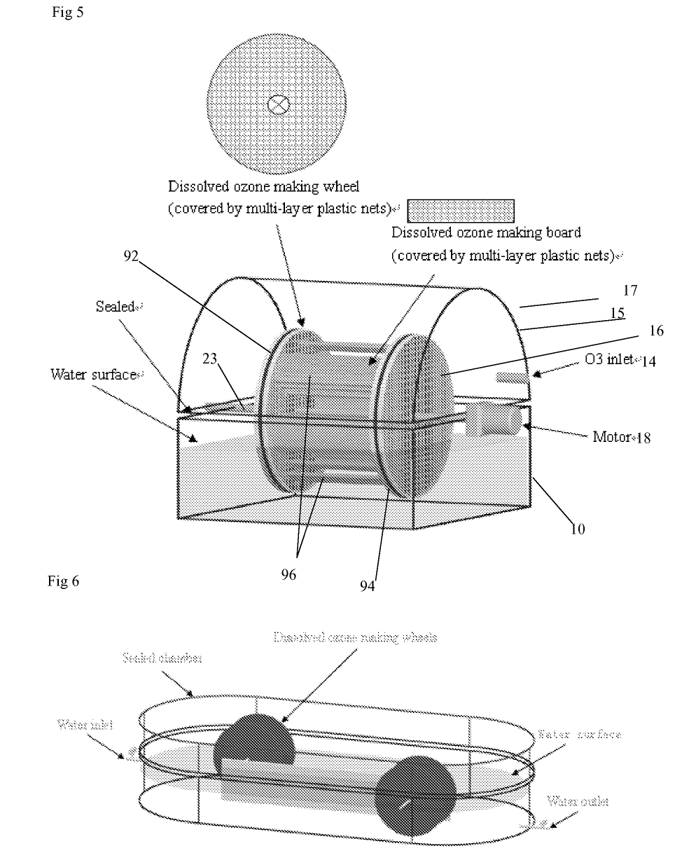

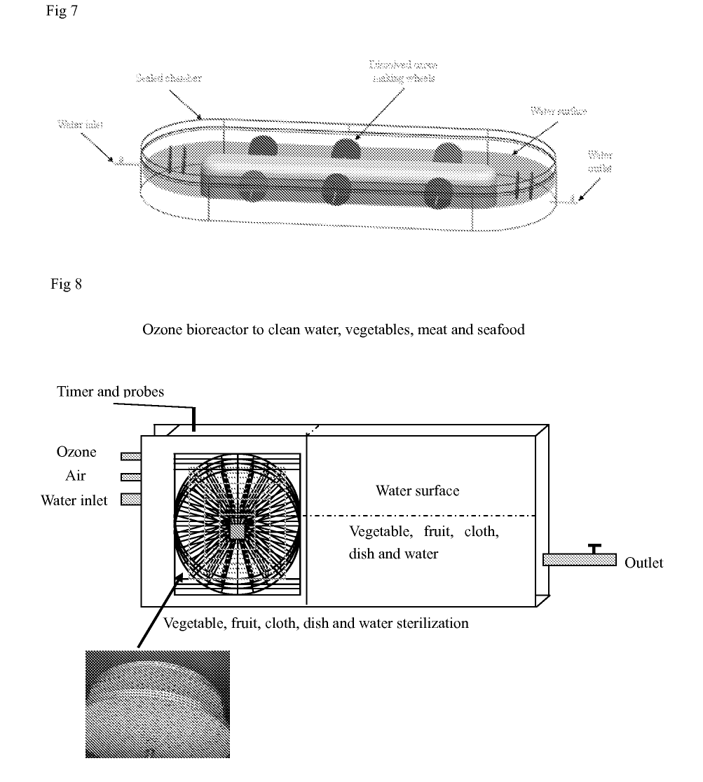

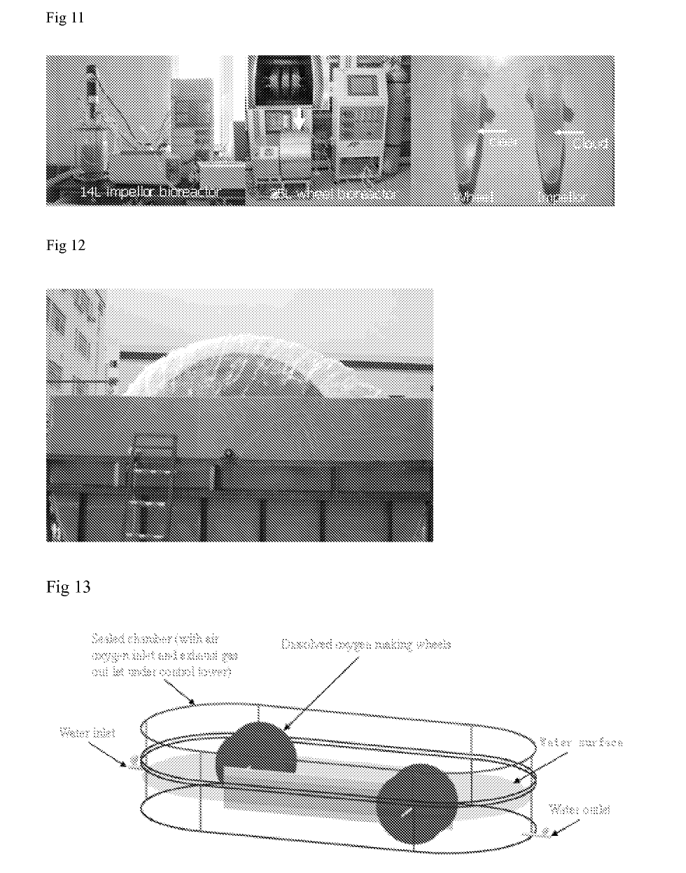

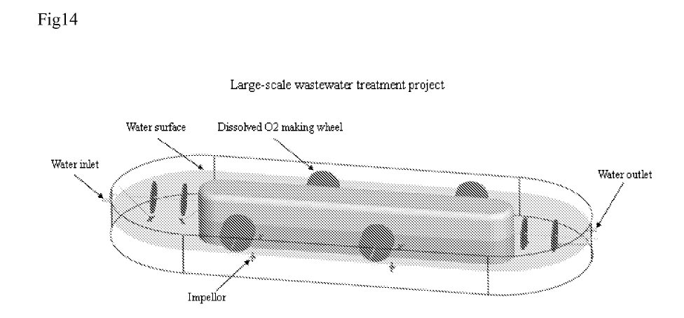

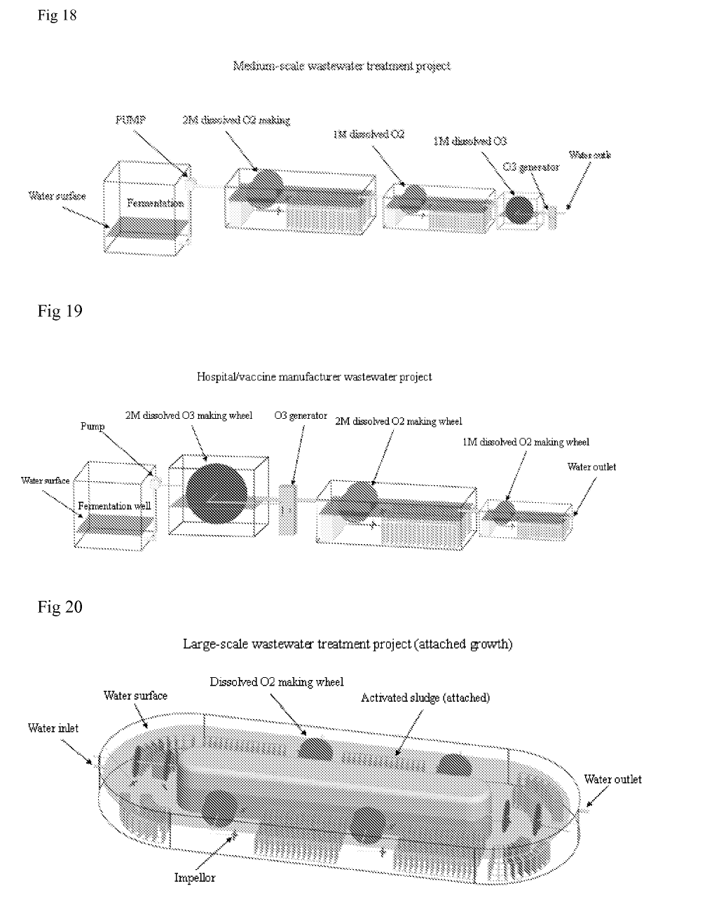

The present invention relates to effectively transferring an ambient gaseous composition (e.g., ambient ozone, ambient air or oxygen, ambient nitrogen, ambient CO₂ and ambient fragrant gases) into a liquid (e.g., water, wastewater and other liquids) by a novel multilayer net-covered rotating wheel method. See FIGS. 1-10. The present invention also relates to novel bioreactors for high-density culture of microorganisms and activated sludge at both suspension and attached status for treatment of water, wastewater and other aerobic and anaerobic fermentation applications. See FIGS. 11-18

Ozone is an effective agent to kill bacteria and viruses. It also oxidizes toxic materials and removes odor and color from water and wastewater. Current water treatment, food or vegetable processing and wastewater treatment often employ ozone-bubbling method to transfer ozone into the water for wastewater treatment. It can not utilize expensive ozone effectively, thus increasing cost and being harmful to its immediate environment. Therefore, this is need for alternatives to transfer ambient ozone directly into water and 100% utilize ozone.

Current biological wastewater treatment or microorganism culture often employs air-bubbling method to transfer oxygen into wastewater for suspension cultivation of activated sludge and microorganisms to improve water quality. The suspension cultivation of activated sludge and microorganisms by air-bubbling method is not an ideal process. For example, biomass of the suspended activated sludge and microorganisms is not higher enough to process large quantity of wastewater by using limited space. Meanwhile, dissolved oxygen level created by the bubbling oxygen transfer method is not good enough to support growth of large biomass of the activated sludge and microorganisms. In addition, mixing force of the bubbling oxygen transfer method is not strong enough to distribute the dissolved oxygen to support large biomass growth of the activated sludge microorganisms. Thus, there is a need for a more effective system to cultivate activated sludge and microorganism at large biomass and a better method to transfer oxygen or to make dissolved oxygen for supporting large biomass growth of the activated sludge microorganisms.

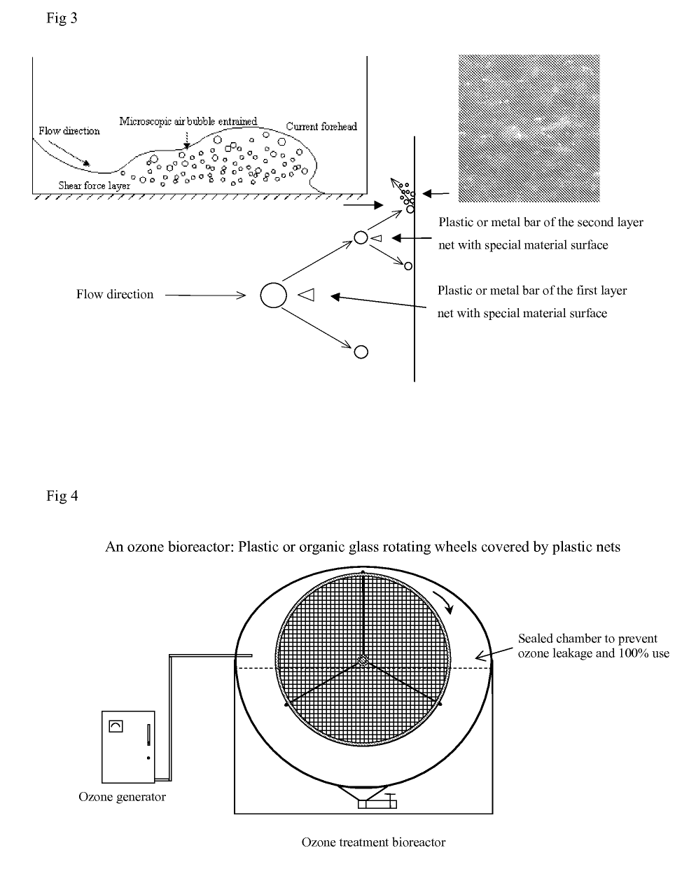

This invention provides a novel system and method for introducing a gaseous composition into a liquid composition as illustrated by FIG. 3. In one example, it includes generation of micro-bubbles of ozone or air by using a gas transfer rotating wheel. This wheel is designed and constructed to have a little less than half surface submerged in a liquid composition and other half surface exposed in ambient gas (such as air or ozone). The wheel is covered by multiple layers of ozone-resistant plastic or metal nets on each sides of the wheel (FIGS. 1, 2, and 5). The gas-exposed portion of the rotating wheel carries ambient gas into a liquid composition in a form of micro-bubbles. The micro-bubbles are generated or entrained through repetitive rounds of medium current hitting on metal or plastic bars of the nets and then surface of the wheel (FIG. 3), and could be detected by dissolved oxygen probe, dissolved ozone probe, high-speed camera probe, Multisizer-3 (Coulter Counter, Beckman), or a phase Doppler anemometer (PDA) probe. The micro-bubble generation rate is related to water current sweeping speed, physical and chemical features of the surface materials, and material surface angle swept. This micro-bubble generation method or mechanism is illustrated in FIG. 3. Examples of these materials include, but are not limited to, polypropylene, or EVA/PE, metal, synthetic glass, and plastics. The material surface physical properties at micrometer and nanometer levels are selected by using scanning electronic microscope (SEM) while their oxygen and ozone transfer properties are experimentally selected by using dissolved oxygen probe, dissolved ozone probe, high-speed camera probe, Multisizer-3 (Coulter Counter, Beckman) or a phase Doppler anemometer (PDA) probe. The method is neither sparging-based nor membrane filtration-based conventional oxygen transfer methods. It involves a novel way to generate or entrain micro-bubbles in water through a multiple layer net covered rotating wheel. DO is scientifically defined as microscopic bubbles in between water molecules.

The systems and methods described herein can be used to transfer ambient oxygen and ozone (which is sealed in a chamber for 100% ozone transfer without leakage) into water or liquid and for low-cost and low-energy treatment of water, and wastewater. Basing on the above method, novel rotating wheel bioreactor systems (FIGS. 4, 5, 6, 7, 8, 9, 10, 11, 12, 13, 14, 15, 16, 17, 18, 19, 20 and 21) were designed and constructed to effectively transfer ambient oxygen (O₂) and ozone (O₃) into water for treatment of water, polluted water, and wastewater. Effective materials on the rotating wheel surface for the best ambient oxygen and ozone transfer (which is sealed in a chamber for 100% ozone transfer without leakage) into water or other liquids were determined through selection of different material and surface nature (chemical and physical nature) by experiments described below.

The present invention also relates to novel bioreactors to high-density culture of microorganisms and culture of activated sludge at both suspension and attached status. Within the scope of the invention are methods to high-density culture of microorganisms and stably culture of activated sludge at larger biomass by using a stacked wall of polymer paper carriers packed in between plastic nets and a novel oxygen transfer method, the method comprised using the just-described system for wastewater treatment.

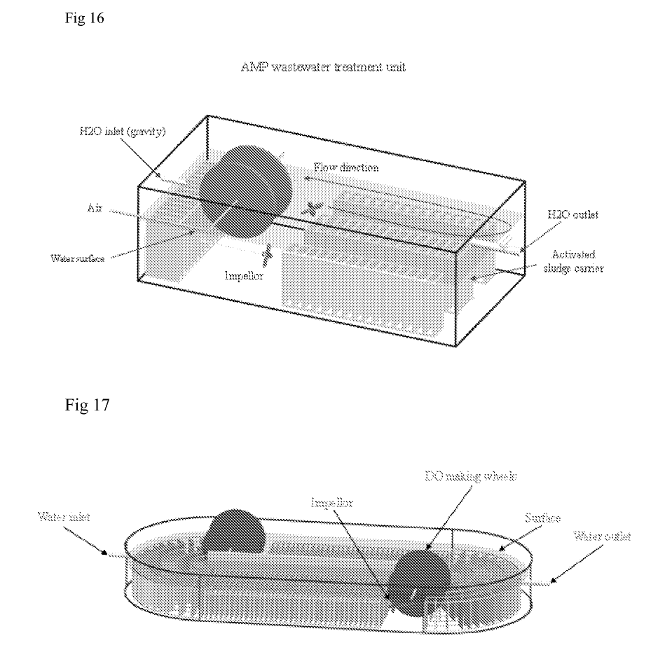

In one example, it includes the use of dissolved oxygen making wheels to culture high-density E-coli in suspension compared with a conventional impellor-based deep tank bioreactor (see FIGS. 11, 13 and 14). In other example, it includes the use of a stacked wall of polymer paper carriers packed in between plastic nets for the stable cultivation of activated sludge with large biomass in attached modes (FIGS. 16-21). Examples of this material include, but are not limited to, non-woven polymer fiber paper carriers and biocompatible plastic nets.

The systems and methods described herein can be used to high-density culture of microorganisms and the activated sludge at both suspension and attached status for treatment of wastewater. Based on the above methods, novel bioreactor systems (see FIGS. 11-21) were designed and constructed to effectively high-density culture of microorganisms and treat wastewater. Effective materials were determined through selection of different material (chemical and physical features) by experiments described below.

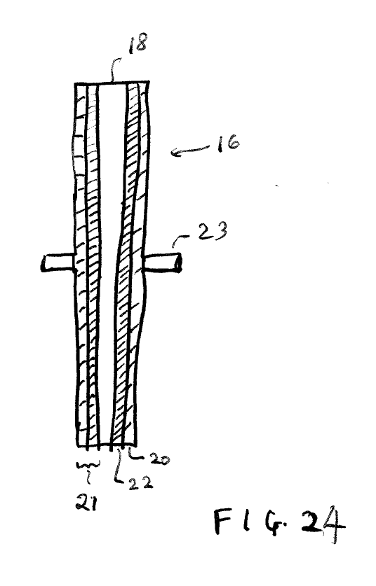

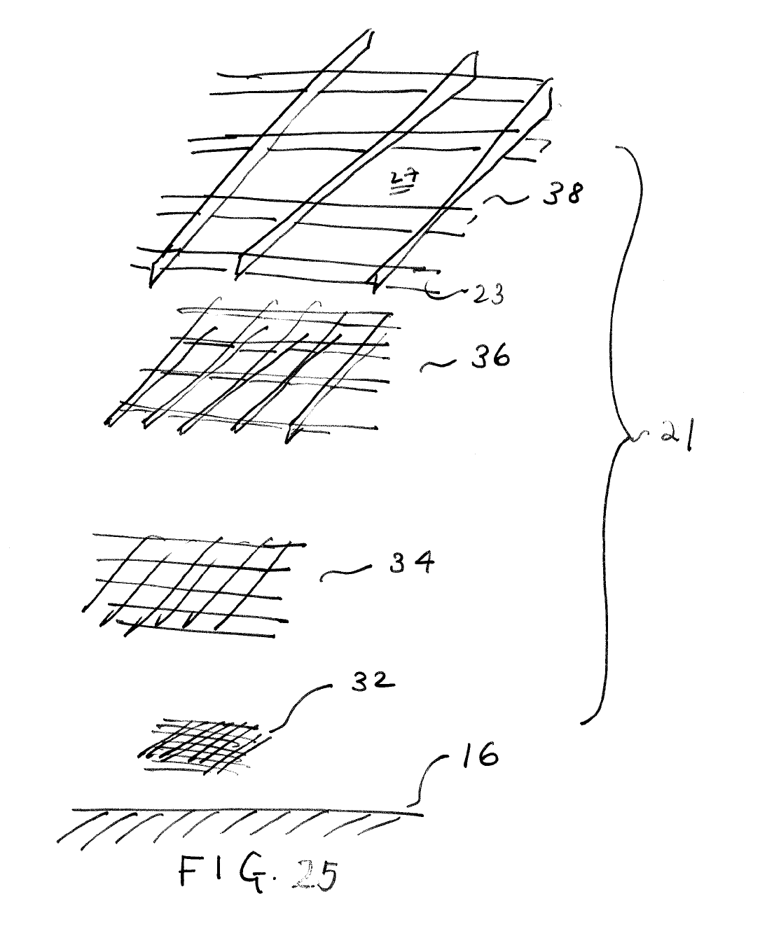

Below are a number of specific embodiments. A system for introducing gas into a liquid medium includes a tank 10, as shown in FIG. 5 or 22. Leading into the tank 10 are a liquid inlet 12, for introducing the liquid medium, and a gas inlet 14, for providing gas to be introduced into the liquid medium. A removable lid 15 covers the tank 10 and defines a sealed chamber 17. The lid 15, shown in its open position in FIG. 23, is closed during operation. As a result, gas introduced via gas inlet 14 is trapped inside the chamber 17.

Referring now to FIG. 5 or 23, a wheel assembly features a rotatable wheel 16 mounted within the tank 10. The wheel 16, which is typically solid aluminum alloy or plastic, is coupled to a motor 18 that spins the wheel 16 at a selected speed. In typical embodiments, the motor 18 spins the wheel 16 between 40 rpm and 90 rpm. The wheel itself typically has a diameter on the order of 0.5 meters. However, the diameter of the wheel and the rotation rate can be made to depend on the specific application.

FIG. 1 lower panel or FIG. 24 shows one example of a wheel 16 from a point of view that faces its rim 18. The wheel spins around an axis 23 that defines a direction orthogonal to the faces of the wheel. The direction along the axis 23 toward the wheel 16 will be referred to as the “wheel-ward” direction. The direction opposite the wheel-ward direction will be referred to as the “anti-wheel-ward” direction.

The wheel 16 has, on one of its faces, a net structure 21 formed by an outer net/tessellation layer 20 and an inner net/tessellation layer 22. Net/Tessellation layers 20, 22, examples of which are shown in FIG. 25, can be formed by ribs 23 that cross over or intersect with each other to define a set of voids. These voids, referred to herein as “cells 27,” form a tessellation of the wheel's face.

The ribs 23 are made of a material that can withstand the effect of the gas present in the chamber 17. Thus, where the gas includes ozone, the ribs 23 are made of an ozone-resistant material. Other materials that can be used for ribs 23 include polypropylene, EVA/PE, synthetic glass, plastic, including ozone-resistant plastic, and metals, such as aluminum.

The cells 27 can be irregular or randomly shaped. However, in some embodiments, the cells 27 have a regular size and shape. In such embodiments, the tessellation layers can be viewed as mesh layers, or nets.

The cells 27 can be square, rectangular, hexagonal, rhombic, or parallelogram. In addition, the cells 27 from different mesh layers need not have the same shape. Thus, one might have an outer layer with rhombic cells and an inner layer with hexagonal cells.

In addition, there can be any number of mesh layers. As discussed in the experiments below, considerable improvement can be achieved with only one mesh layer. However, the dissolution rate increases as more layers are added.

As shown in FIG. 1 or 24, there are only two layers. However, the number of layers is not limited to two. For example, wheels 16 with three to six layers can also be used. Moreover, the innermost layer, i.e. the layer closest to the wheel 16 can be integral with the wheel 16 itself. For example, the wheel 16 could have an array of holes drilled into it to form a mesh, or the wheel 16 could have an array of depressions formed into it.

FIG. 25 illustrates an exploded view a net structure 21 formed by a stack of layers beginning with a wheel-ward-most layer 32 on the surface of the wheel 16, and additional layers 34, 36, 30 stacked in the anti-wheel-ward direction.

Although the stacking of mesh layers, or tessellation layers, on a wheel 16 is a useful way to construct the net structure 21, the net structure 21 can be constructed in any other way, for example by machining, casting, or etching.

In the particular case of a wheel with only two tessellation layers, as shown in FIG. 1 or 24, the cell size of the wheel-ward least, or outermost layer is preferably larger than the cell size of the wheel-ward most, or innermost layer. In cases where more than two layers are present on the wheel 16, the cell sizes preferably decrease as one proceeds in the wheel-ward direction. As used herein, cell size refers to a metric representing how large a cell 27 is. Suitable metrics include cell area, cell perimeter, or the length of a cell's side.

In operation, the tank 10 is partially filled with the liquid into which the gas is to be introduced. The level to which the tank 10 is filled is such that the wheel 16 partially protrudes above the surface of the liquid. Preferably, as much as half of the wheel 16 is above the liquid's surface. The chamber 17 is then filled with the gas. After the chamber 17 is filled, the motor 18 rotates the wheel 16 at a pre-defined rate for some pre-defined period.

Referring to FIG. 25, as the wheel 16 spins, cells 27 from the net structure 21 on the wheel 16 are alternately plunged beneath and raised from the liquid surface. The physical mechanism by which the net structure 21 on the wheel 16 hastens the dissolution of gas is not completely understood. It is believed, however, that as the wheel 16 spins, bubbles of ambient gas become entrained in outermost layer 38 of the net structure 21. These bubbles potentially migrate wheel-ward through the net structure 21, into smaller cells 27 in an inner layer 36. In doing so, they encounter the ribs 23 of the inner layer 36 and are divided by those ribs 23 into smaller bubbles. This wheel-ward migration through the net structure, which results in progressively smaller bubbles, would proceed until one reached the wheel-ward most, or innermost layer 32. Micro-bubbles formed by the wheel-ward most layer 32 escape and are small enough to be easily dissolved. However, the foregoing physical mechanism is only a theory and is not to be used as a basis for limiting the scope of the claims. As discussed below, regardless of the physical mechanism relied upon, the apparatus disclosed herein rapidly and efficiently dissolves gas in liquid.

In some practices, gas is continuously fed into the chamber 17 so that a constant concentration or amount of gas is always present.

As the optimal level of dissolved gas is reached, the supply of gas is cut off, and the wheel 16 is allowed to spin for some time thereafter. During this period, whatever residual gas is in the chamber 17 dissolves in the liquid. This enables almost 100% utilization of the gas, depending on how long the wheel 16 is kept spinning after the gas supply has been cut off.

In one example, wheels having a 0.5 meter diameter and different numbers of tessellation layers were immersed in 110 liters of water in a chamber filled with oxygen. It was found that with no layers, it would take 20 minutes to reach a dissolved oxygen level of 100% from a baseline dissolved oxygen level of 0%. Adding a net structure with one layer to the wheel 16 reduced this time a mere 150 seconds. Adding a second layer to the net structure reduced the time further, to only 90 seconds.

In another experiment, the chamber was filled with 110 liters of water tinted by a blue ink, and the remaining portion of the chamber was filled with ozone. The same wheel diameter (0.5 meters) and rotation rate (90 rpm) was used. In this case, the water turned clear in 40 minutes when the wheel 16 had one tessellation layer, and turned clear in 25 minutes when a net structure with two tessellation layers was used.

The effectiveness of having a net structure with multiple tessellation layers can be seen in, e.g., Table 3 in Example 3 below, which shows the level of dissolved oxygen during the first six minutes of operation using different numbers of tessellation layers. In this experiment, the tessellation layers were made of aluminum, the wheel 16 was 1.0 m in diameter and spun at 53 rpm, and there were 650 liters of a 0.16 g/L aqueous solution of Na₂SO₃.

Gas dissolution assisted by net structures as described herein can be used in a variety of applications. For instance, the system can be used to introduce oxygen into waste-water, thus facilitating growth of aerobic bacteria. Or, the system can be used to disinfect water with ozone. Such water is useful for washing, and thereby disinfecting, vegetables or meats. Or the system can be used to introduce fragrant gas, for example a gas carrying a lemon scent, into water, or to introduce chlorine or other disinfectant gases into swimming pool water. Ozone treatments as described herein can also be used to pre-treat contaminated wastewater, such as phenol contaminated waste water.

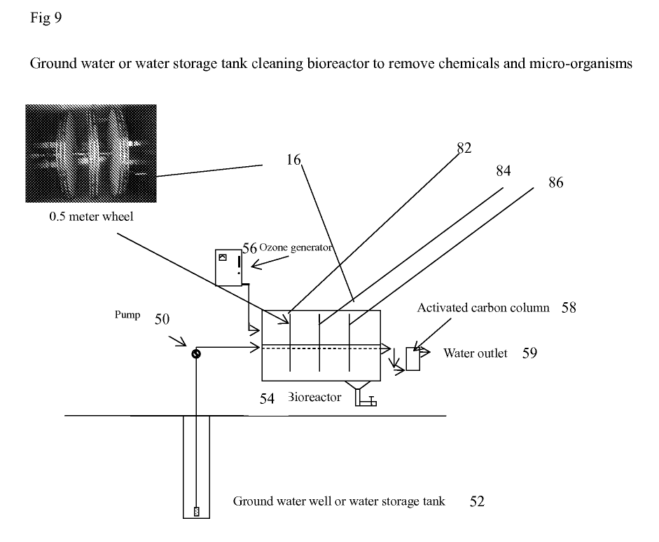

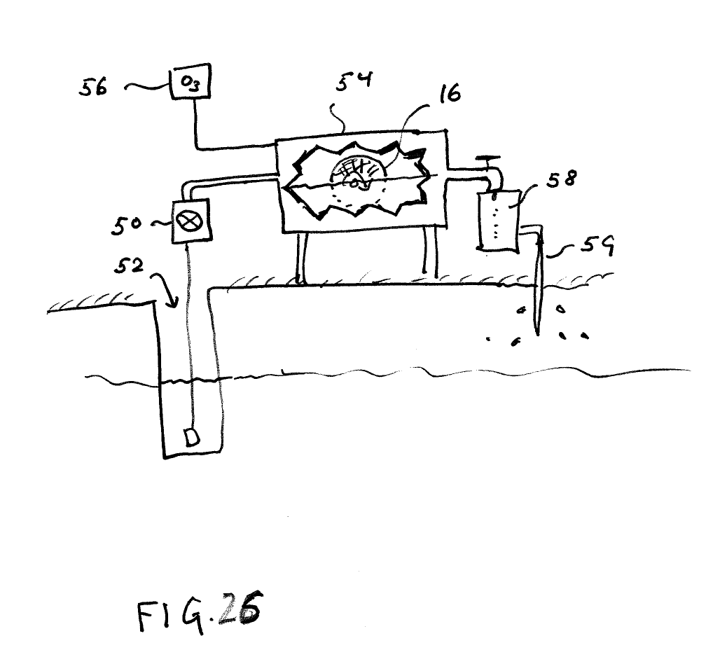

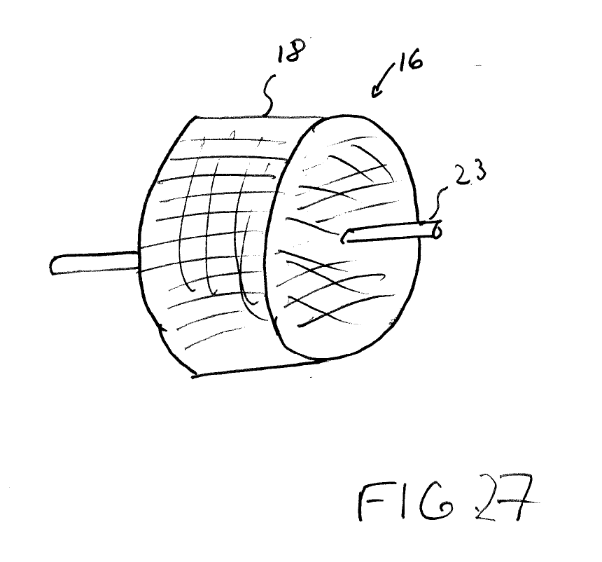

An ozone treatment as described herein can also be used to treat contaminated ground water, as shown in FIG. 9 or 26. The illustrated system includes a pump 50 for drawing ground water from a well 52, and a bio-reactor 54 having a 0.5 m diameter wheel 16. An ozone generator 56 provides ozone for filling the bioreactor 54. Water from the bioreactor 54 is passed through an activated carbon column 58 before being discharged back into the ground at a water outlet 59.

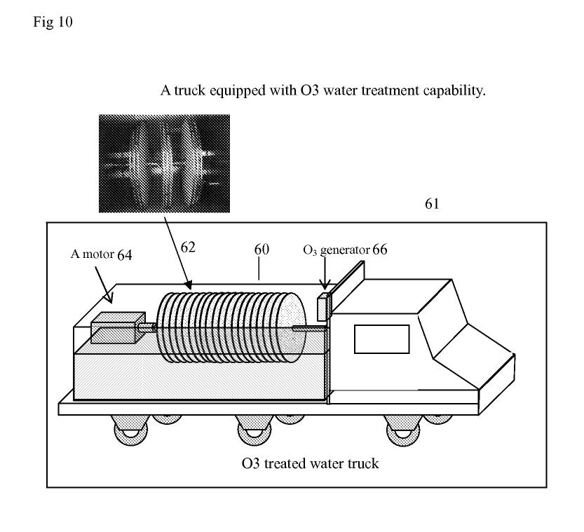

A bioreactor as described herein is sufficiently small and portable to be transported to a site on a truck 60, as shown in FIG. 10. In the illustrated bioreactor 61, a motor 64 drives a wheel assembly having an array of wheels 62, each of which is constructed as described in connection with FIG. 5 or 23. This provides more rapid dissolution of ozone generated by an ozone generator 66. A portable system as shown in FIG. 10 is particularly useful for tasks such as groundwater remediation, or decontaminating swimming pools, ponds, and other bodies of water that are not easily transportable.

The rate at which the wheel 16 introduces gas into a liquid depends on the structure of the wheel 16 and its accompanying net structure 21. Example 15 shows the extent to which several 0.25 m diameter wheels can dissolve oxygen within 7 minutes when rotated at 90 rpm.

Systems and methods as described herein can be adapted to facilitate high-density culture of microorganisms and culture of activated sludge, whether suspended or attached. For example, the system can be provided with a stacked wall of polymer paper carriers packed between plastic meshes, Examples of such a material include, but are not limited to, non-woven polymer fiber paper carriers and biocompatible plastic nets.

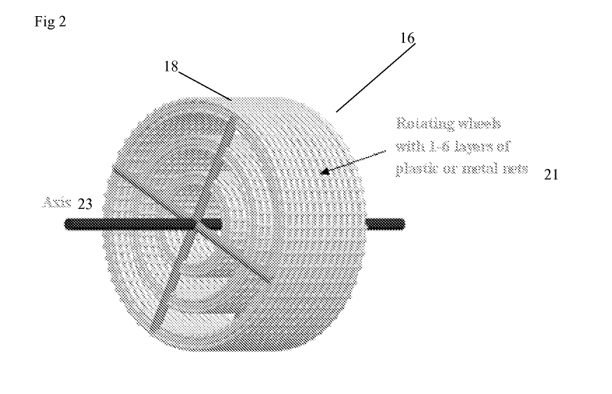

In additional embodiments, the rim 18 of the wheel 16 defines a circumferential face that can have a net structure 21 disposed thereon. This is particularly useful for wheel having a wide rim 18, such as that shown in FIG. 2 or 27. The net structure 21 is as described above in connection with the net structures placed on those faces of the wheel whose normal vector is parallel, rather than perpendicular, to the axis 23. Since the wheel-ward and anti-wheel-ward directions are defined with respect to a face of the wheel, in the context of FIG. 2 or 27, where the face is in fact the wheel's rim 18, the sizes of the cells 27 in the net structure 21 would again decrease as one proceeds in the wheel-ward direction.

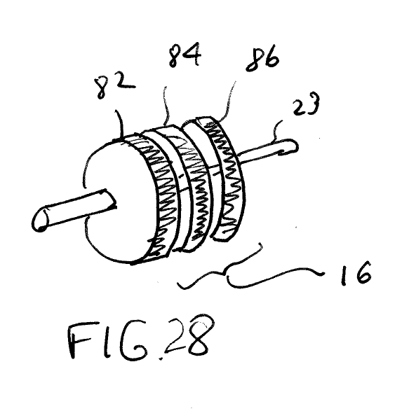

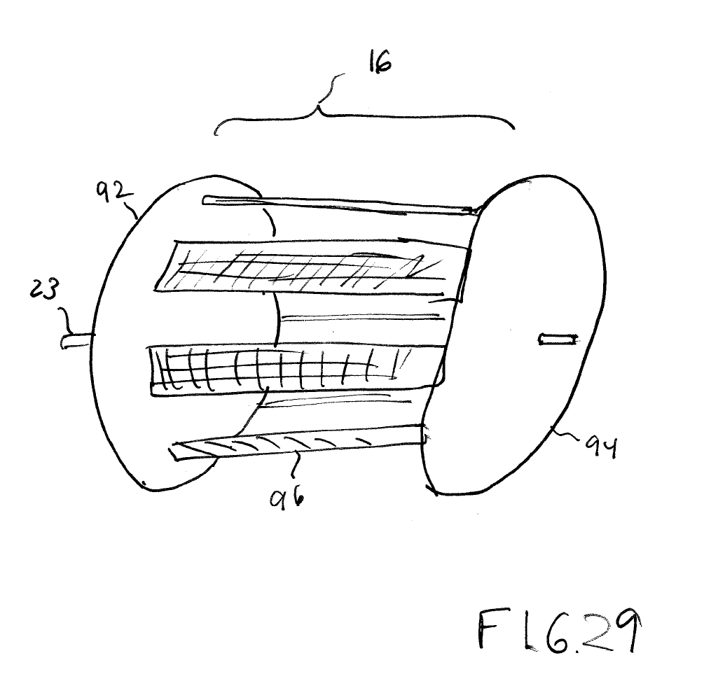

The expansion of wheel surface area devoted to a net structure, as shown in FIG. 2 or 27, can also be achieved using a wheel assembly having a plurality of co-axial wheels 82, 84, 86, as shown in FIG. 9 or 28.

FIG. 5 or 29 shows another way to increase the wheel area devoted to a net structure 21. In FIG. 5 or 29, the wheel assembly includes first and second coaxial wheels 92, 94 with boards 96 extending between them. A net structure 21 is disposed on the boards 96. Each board 96 can be mounted such that a vector normal to the face of that board 96 is parallel with a radial vector (i.e., a vector normal to the axis 23 and extending toward the board 96). However, a board can also be mounted such that a vector normal to the face of that board forms a radial vector.

Moreover, in the embodiment shown in FIG. 5 or 29, one or more boards 96 can be moved radially outward such that a portion of a board 96 extends beyond the rim of the wheels 92, 94. This results in a paddle-wheel arrangement, and also permits the wheels 92, 94 to be mounted above the surface of the water such that only the boards 96 dip below the surface. Such a configuration is useful when the tank 10 is too shallow to accommodate the wheels 92, 94.

The configurations shown in FIGS. 27-29 are particularly useful because the linear velocities of the cells 27 do not vary with the location, as they would where the net structure 21 is on a side face (i.e. a face whose normal vector is parallel to the axis 23) of the wheel.

In FIG. 23, only a single wheel 16 is shown. However, any number of wheels can be used. Moreover, the wheels can be placed at varying locations within the tank 10.

Moreover, as described herein, the net structure 21 is plunged into and out of the water by mounting it on a rotating structure. However, other mechanisms for plunging the net structure 21, or portions thereof, are possible. For example, the net structure 21 can be mounted on a reciprocating structure, such as a flat or curved board, that is repetitively plunged into and out of the water. Or the net structure 21 can be mounted on an endless belt that is looped between a rotating cylinder below the surface and another rotating cylinder above the surface.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Further, any mechanism proposed below does not in any way restrict the scope of the claimed invention.

### Example 1

In this example, a modified bicycle wheel structure was used for the O₂ transfer wheel construction as shown in FIGS. 1 and 2. The results indicated that this design was successfully used to make the strongest and the most stable gas transfer wheel. For example, it led to the lightest wheel structure without losing the wheel stability. For other example, it led to the most stable wheel structure having minimum water resistance while rotating (FIGS. 1, 2, 4, 5, and 8-12).

### Example 2

In order to generate micro-bubbles, we hypothesized that large air bubbles becomes micro-bubbles after 3 times of hitting by and interaction with (entraining microscopic bubbles during the interaction) metal or plastic or organic glass bars or surface of the nets and finally the solid surface (illustrated in FIG. 3). To examine this hypothesis, we used a solid wheel covered with 0-2 layers of plastic or organic glass or metal nets (0.5 meter diameter wheel employed). The microscopic bubble formation was detected by dissolved oxygen (DO) probe or high-speed camera or a phase Doppler anemometer (PDA). To transfer O₂, we used aluminum nets. Results in Table 1 clearly show that the 2-layer net covered wheel had significantly better oxygen transfer capability.

TABLE 1
Oxygen transfer.

| | | Rotation | | | Minutes required |
|---|---|---|---|---|---|
| Wheel | Wheel | speed | Net | Volume | to reach DO 100% |
| diameter | numbers | (rpm) | layer | treated | from baseline DO 0% |
| 0.5 meter | 2 | 90 | 0 | 110 liters | 20 |
| 0.5 meter | 2 | 90 | 1 | 110 liters | 2.5 |
| 0.5 meter | 2 | 90 | 2 | 110 liters | 1.5 |

To transfer O₃, we used plastic nets to avoid ozone's oxidization activity. We used ozone's capability to remove ink's blue color as a marker. Results in Table 2 indicate that the 2-layer net covered wheel had better ozone transfer capability.

TABLE 2
O₃ transfer

| | | Rotation | | | Minutes required |
|---|---|---|---|---|---|
| Wheel | Wheel | speed | Net | Volume | to remove ink's |
| diameter | numbers | (rpm) | layer | treated | blue color from water |
| 0.5 meter | 2 | 90 | 0 | 110 liters | N/A |
| 0.5 meter | 2 | 90 | 1 | 110 liters | 40 |
| 0.5 meter | 2 | 90 | 2 | 110 liters | 25 |

### Example 3

As mentioned above, large air bubbles becomes micro-bubbles after 3 times of hitting on metal or plastic or organic glass bars of the nets and finally the solid surface (see FIG. 3). To further examine this hypothesis, we used a solid wheel covered with 1-6 layers of plastic or organic glass or metal nets. The microscopic bubble formation was detected in the same manner described above. To transfer O₂, we used aluminum nets. Results in Table 3 and Table 4 clearly indicate that 6-layer net covered wheel had significantly better oxygen transfer capability.

For the results shown in Table 3, a wheel of 1.0 meter diameter was used. Oxygen transfer is expressed by DO(%). Total volume of water is 650 L; and 0.16 g/L of Na₂SO₃ was loaded. The wheel rotation speed was 53 rpm.

TABLE 3

| | # of Layers | | | | |
|---|---|---|---|---|---|
| Minutes | 6 | 4 | 3 | 2 | 1 |
| 0 | 0 | 0 | 0 | 0 | 0 |
| 1 | 69.1 | 69.4 | 48.6 | 42.5 | 29.2 |
| 2 | 90.0 | 84.1 | 68.9 | 65.0 | 50.5 |
| 3 | 95.0 | 87.2 | 75.1 | 72.3 | 63.0 |
| 4 | 95.5 | 89.1 | 76.9 | 77.4 | 70.1 |
| 5 | 96.1 | 90.5 | 78.0 | 78.7 | 72.5 |
| 6 | 96.0 | 91.3 | 78.5 | 78.8 | 72.5 |

For the results shown in Table 4, a wheel of 1.0 meter diameter was used. The oxygen transfer speed was expressed by K_{LA}. Total volume of water was 650 L. 0.16 g/L of Na₂SO₃ is loaded. The wheel rotation speed was 53 rpm.

TABLE 4

| # of Layers | K_{La} (min⁻¹) | R² | q_{c} (kg/h) | E (kg/kW h) |
|---|---|---|---|---|
| 6 | 1.3500 | 0.9839 | 0.43065 | 0.468098 |
| 4 | 1.1121 | 0.9645 | 0.397576 | 0.361433 |
| 3 | 1.0731 | 0.9987 | 0.383633 | 0.348758 |
| 2 | 0.9405 | 0.9744 | 0.336229 | 0.305663 |
| 1 | 0.7543 | 0.9497 | 0.269662 | 0.245148 |

### Example 4

A wheel bioreactor assembly that had a wheel of 2.0M diameter was employed for O₂ and O₃ transfer. As shown in Table 5, the results indicated similar O₂ and O₃ transfer speeds.

TABLE 5

| | | | | | Minutes | Minutes |
|---|---|---|---|---|---|---|
| | | | | | required | required |
| | | | | | to reach | to remove |
| | | | | | DO 100% | ink's |
| | | Rotation | | Volume | from | blue color |
| Wheel | Wheel | speed | Net | treated | baseline | from |
| diameter | numbers | (rpm) | layer | (L) | DO 0% | water |
| 2.0 meter | 2 | 30 | 0 | 4500 | N/A | N/A |
| 2.0 meter | 2 | 30 | 1 | 4500 | 15 | 60 |
| 2.0 meter | 2 | 30 | 2 | 4500 | 9 | 35 |

### Example 5

In order to 100% transfer the environmental O₃ into a sealed chamber for waster sterilization (Table 6), vegetable wash (Table 7), dish (Table 8) (ozone 40 mg/L), and meat cleaning (ozone 40 mg/L), we stopped wheel rotation 10 minutes after stopping O₃ supply. Our gas O₃ measurement indicated that no O₃ exist after 10 minutes of rotation after O₃ supply stopped. This indicated that all the O₃ was 100% transferred into the water without leakage to the surrounding environment.

TABLE 6
O₃ treatment of tap water

| | Sample dilution time | | |
|---|---|---|---|
| | 0x | 10x | 100x |
| Before treatment | Too many unable | Too many unable | 61 |
| | to count | to count | |
| 10 mg/L O₃ loaded | 16 | 0 | 0 |
| 20 mg/L O₃ loaded | 10 | 0 | 0 |
| 30 mg/L O₃ loaded | 0 | 0 | 0 |

TABLE 7
O₃ treatment of vegetables

| | Sample dilution time | | |
|---|---|---|---|
| | 10x | 100x | 1000x |
| Before treatment | Too many unable to count | 75 | 9 |
| 10 mg/L O₃ loaded | Some unable to count | 0 | 0 |
| 20 mg/L O₃ loaded | Some unable to count | 0 | 0 |
| 30 mg/L O₃ loaded | 1 | 0 | 0 |
| 40 mg/L O₃ loaded | 0 | 0 | 0 |

TABLE 8
O₃ treatment of dishes

| | Sample dilution time | | |
|---|---|---|---|
| | 10x | 100x | 1000x |
| Before treatment | Too many unable to count | 75 | 9 |
| 10 mg/L O₃ loaded | Some unable to count | 0 | 0 |
| 20 mg/L O₃ loaded | Some unable to count | 0 | 0 |
| 30 mg/L O₃ loaded | 1 | 0 | 0 |
| 40 mg/L O₃ loaded | 0 | 0 | 0 |

### Example 6

The above-described ambient gas transfer bioreactor was used to transfer fragrant gas (lemon scented) into water. The results (shown in Table 9 below) indicated rapid addition of fragrant gas (lemon scented) into the water.

TABLE 9

| Time | Control | 0.5 hour | 1.0 hour | 2.0 hour | Tap water |
|---|---|---|---|---|---|
| Lemon | None | Very mild | Mild | Strong | None |
| scent | | | | | |

### Example 7

The above-described bioreactor (illustrated in FIG. 4), which can achieve 100% O₃ transfer, was used for treatment of fish pond water. After the O₃ treatment (6-12 mg/L O₃), significant clear pond water was observed.

### Example 8

The above-described bioreactor (illustrated in FIG. 5) was also employed for treatment of swimming pool water. After the O₃ treatment (6-12 mg/L O₃), significant clear swimming pool water was observed.

### Example 9

The above-described bioreactor (illustrated in FIG. 5) was used for nitrogen and CO₂ transfer in order to pre-treat culture medium for bacterial seed inoculation of an anaerobic fermentation. Our result indicated 15 minutes of treatment time was enough to lower dissolved oxygen in the medium to 0-2%, indicating an valuable application of pre-treatment of anaerobic fermentation culture media.

### Example 10

One of industrial wastewater treatment examples (East-China Pharma) was described in this example. The above-described bioreactor (illustrated in FIG. 4) was used to pre-treat chemically (phenol) contaminated wastewater in order to alter the chemical composition for more efficient biological method treatment. Table 10 shows the results, indicating the pre-treatment of the wastewater increased efficiency of next step biological treatment. This sets up a foundation for the ozone pre-treatment of the chemically contaminated industrial wastewater by using the close to 100% ozone-use method.

TABLE 10

| | | | COD | COD after 12 | |
|---|---|---|---|---|---|
| COD of | Ozone | COD after | Before | hours of | COD |
| the original | pre- | ozone pre- | biological | biological | reduction |
| samples | treatment | treatment | treatment | treatment | % |
| 2331 | No | | 2331 | 1792 | 23 |
| 2331 | Yes | 1658 | 1658 | 638 | 62 |

### Example 11

The above-described bioreactor (illustrated in FIG. 4) was used for treatment of industrially contaminated pond water. The results are shown in Table 11 below. As shown in Table 11, the ambient ozone transfer bioreactor effectively removed odor and color from the industrially contaminated colored pond water. This result also indicated effective transfer of ambient ozone into the water at low-cost by the ambient ozone transfer bioreactor.

TABLE 11

| Time | Control | 1.0 hour | 2.0 hour | Tap water |
|---|---|---|---|---|
| Odor | Very strong | Very mild | None | None |

### Example 12

Water samples were obtained from the algae-bloomed Dianchi Lake, the sixth largest freshwater lake in China. The water samples were then subject to 30 minutes of ozone treatment using the above-described bioreactor. It was found that no taste, odor and color were observed after the treatment.

### Example 13

Ozone transfer speed into water was examined by using an ambient ozone transfer bioreactor (6 liter work volume; 0.25 meter in diameter wheels x2; 12 g/hour O₃ generator). Table 12 shows the results.

TABLE 12

| | Time | | |
|---|---|---|---|
| Item | 0 minute | 20 minute | 40 minute |
| Ozone (mg/L) | 0.0 ± 0.0 | 10.2 ± 1.5 | 18.2 ± 1.6 |

### Example 14

Ozone transfer effect was examined by using an ambient ozone transfer bioreactor (6 liter work volume; 0.25 meter in diameter wheels x2; 12 g/hour O₃generator) and ink colored water samples. Our result showed that O₃transfer effect was clearly shown by de-colored water samples at various treatment periods.

### Example 15

In order to find the best materials for constructing the rotating wheels, different materials including metal, plastic, polymer, synthetic glass in different structural shapes were studied by scanning electronic microscope (SEM), dissolved oxygen probe, dissolved ozone probe, high-speed camera probe and a phase Doppler anemometer (PDA) probe for their oxygen and ozone transfer properties. Table 13 and Table 14 show their oxygen and ozone transfer results. The synthetic glass wheel coated with stainless steel metal nets at both sides was chosen for current use. For this study, wheels with 0.25 meter in diameter (90 rpm) were used in wastewater samples.

TABLE 13

| | The highest |
|---|---|
| | dissolved |
| | oxygen level |
| Wheel & construction material with | (%) reached |
| different physical and chemical features | in 7 minutes |
| Synthetic glass wheel with holes drilled halfway through | 86.1 |
| (not completely penetrating) | |
| Synthetic glass wheel completely covered on both sides | 56.3 |
| by non-woven polymer paper | |
| Synthetic glass wheel with narrow plastic boards (w/ | 77.4 |
| small drilled penetrating holes) glued to the surface | |
| Synthetic glass wheel with narrow aluminum boards (w/ | 82.0 |
| many small drilled penetrating holes) glued to the | |
| surface | |
| Wheels made out of 4 layers of stainless steel mesh (0.5 | 86.5 |
| cm diameter square holes) | |
| Synthetic glass wheel completely covered on both sides | 100.2 |
| with stainless steel mesh (0.5 cm diameter square holes) | |
| Synthetic glass wheel completely covered on both sides | 101.8 |
| by plastic mesh (0.5 cm diameter square holes) | |
| Synthetic glass wheel completely covered on both sides | 102.0 |
| by aluminum mesh (0.5 cm diameter diamond holes) | |

TABLE 14

| | Time (minutes) | The |
|---|---|---|
| | required | highest |
| | to reach | dissolved |
| | the highest | ozone |
| Wheel & construction material with | dissolved | level |
| different physical and chemical features | ozone level | (mg/L) |
| Synthetic glass wheel with holes drilled | 46 | 1.5 |
| halfway through (not completely | | |
| penetrating) | | |
| Synthetic glass wheel completely covered | 42 | 1.6 |
| on both sides by non-woven polymer paper | | |
| Synthetic glass wheel with narrow plastic | 23 | 11.8 |
| boards (w/small drilled penetrating holes) | | |
| glued to the surface | | |
| Synthetic glass wheel with narrow | 21 | 8.7 |
| aluminum boards (w/many small drilled | | |
| penetrating holes) glued to the surface | | |
| Wheels made out of 4 layers of stainless | 40 | 15.3 |
| steel mesh (0.5 cm diameter square holes) | | |
| Synthetic glass wheel completely covered | 32 | 16.1 |
| on both sides with stainless steel mesh (0.5 | | |
| cm diameter square holes) | | |
| Synthetic glass wheel completely covered | 30 | 16.0 |
| on both sides by plastic mesh (0.5 cm | | |
| diameter square holes) | | |
| Synthetic glass wheel completely covered | 28 | 16.0 |
| on both sides by aluminum mesh (0.5 cm | | |
| diameter diamond holes) | | |

### Example 16

In order to understand if the rotating wheel (1.0 meter in diameter) oxygen transfer method and air-sparging interfered with each other or if they employ different mechanisms, different methods and combinations were employed. The results in Table 15 indicated that these two method used together as a combination did not perform better than the rotating wheel method alone, suggesting their interference on each others' performance. This also suggests different mechanisms of the two oxygen transfer methods.

TABLE 15

| | Percent (%) dissolved oxygen |
|---|---|
| Oxygen transfer method | after 10 minute treatment |
| Air-sparging | 79 |
| Rotating wheel | 90 |
| Air-sparging + rotating wheel | 92 |

### Example 17

Rotating wheels with different diameters (0.25, 0.5, 1.0, or 2.0 meter) were studied for their ozone transfer properties in periods of 10 minutes. Table 16 shows the results, indicating that all sizes of the diameters worked well. For all the experiments, a 12 g/L O₃ generator was used. Thus, the ozone supply was not enough to supply O₃ to the large wheel ozone transfer experiments due to the ozone generator's capability limitation.

TABLE 16

| | Ozone (mg/L) after 10 |
|---|---|
| Diameter (meter) | minutes of the transfer |
| 0.25 | 15.5 |
| 0.5 | 8.6 |
| 1.0 | 5.6 |
| 2.0 | 2.6 |

### Example 18

The above-described O₃ transfer technology was also applied to treat contaminated ground water. Detailed design was shown in FIG. 9. The results indicated a significantly reduced bacterial level from 600 to 0 colonies by using 10 mg/L O₃ supply. Other chemical contaminants were also studied. Significant common chemicals for agriculture purposes were reduced to zero by using 10 mg/L O₃ supply dosage.

The present invention also relates to novel bioreactors to high-density culture of microorganisms and culture of activated sludge at both suspension and attached status. Within the scope of the invention are methods to high-density culture of microorganisms and stably culture of activated sludge at larger biomass by using a stacked wall of polymer paper carriers packed in between plastic nets and a novel oxygen transfer method, the method comprised using the just-described system for wastewater treatment.

In one example, it includes the use of dissolved oxygen making wheels to culture high-density E-coli in suspension compared with a conventional impellor-based deep tank bioreactor (FIGS. 11, 13 and 14). In other example, it includes the use of a stacked wall of polymer paper carriers packed in between plastic nets for the stable cultivation of activated sludge with large biomass in attached modes (FIGS. 16, 17, 18, 19, 20 and 21). Examples of this material include, but are not limited to, non-woven polymer fiber paper carriers and biocompatible plastic nets.

The systems and methods described herein can be used to high-density culture of microorganisms and the activated sludge at both suspension and attached status for treatment of wastewater. Based on the above methods, novel bioreactor systems (FIGS. 11, 12, 13, 14, 15, 16, 17, 18, 19, 20 and 21) were designed and constructed to effectively high-density culture of microorganisms and treat wastewater. Effective materials were determined through selection of different material (chemical and physical features) by experiments described below.

### Example 19

FIG. 11 shows an experiment to compare oxygen transfer speeds of DO making wheel bioreactor and a conventional impellor/bubbling-based deep tank bioreactor. Air was used for the culture. Maximum speed of the impellor was 750 rpm while maximum rotating speed of the wheel was 190 rpm. The result (FIG. 11, panel 3) clearly suggested that DO making wheel bioreactor generated significantly less shear force, namely much clear supernatant of the sample after centrifugation. Results in Table 17 indicated similar maximum oxygen transfer speeds were obtained by two bioreactors with two different oxygen transfer mechanisms. Results in Table 18 indicated similar maximum cell densities were obtained by two bioreactors with two different oxygen transfer mechanisms. Results in Table 19 indicated that DO making wheel bioreactor did not significantly affect cell viability and generated much less shear force than that of impellor-based deep tank bioreactor. Taken together, DO making wheel bioreactor is characterized by less shear force generated, excellent oxygen transfer speed and cell growth feature when compared with conventional impellor/bubbling-based deep tank bioreactor). We concluded that DO making wheel is a novel effective oxygen transfer method as illustrated by FIG. 3.

TABLE 17
Comparison of the maximum oxygen transfer
speeds of two bioreactors.

| Culture period (hour) | 1 | 2 | 3 | 6 | 10 |
|---|---|---|---|---|---|
| DO (wheel) | 75% | 45% | 41% | 52% | 2% |
| DO (impellor + bubbling) | 70% | 50% | 45% | 52% | 2% |

TABLE 18
Comparison of the maximum cell densities (OD) of two bioreactors.

| Culture period (hour) | 1 | 2 | 3 | 4 | 6 | 9 |
|---|---|---|---|---|---|---|
| OD (wheel) | 1.0 | 1.8 | 2.7 | 3.5 | 4.6 | 19.2 |
| OD (impellor + bubbling) | 1.2 | 2.1 | 3.3 | 4.2 | 5.2 | 20 |

TABLE 19
Comparison of the shear force (cell viabilities) generated
by two bioreactors

| Culture period (hour) | 1 | 2 | 3 | 4 | 6 | 9 |
|---|---|---|---|---|---|---|
| Cell viability % (wheel) | 98 | 98 | 96 | 95 | 95 | 95 |
| Cell viability % | 89 | 68 | 46 | 24 | 4 | 0 |
| (impellor + bubbling) | | | | | | |

### Example 20

A method for adaptation of activated sludge is described in this example. Wastewater COD 400-800 mg/L was used in a container supplied with air sparging at room temperature (18-24° C.). After one week of cultivation, the activated sludge appeared. Then, 50% of the wastewater was removed and the same volume of fresh wastewater volume added every week. In two weeks, qualified activated sludge was obtained. To maintain the activated sludge cultivation, 50% volume change every week continued.

### Example 21

A bioreactor with 60-gram of a stacked polymer paper per liter of wastewater was used to treat wastewater. The wastewater was cultivated for 6, 8, 10 and 12 hours. COD was measured. Results in Table 20 showed effective removal of COD was accomplished.

TABLE 20

| Wastewater | | Wastewater | Paper carrier | COD |
|---|---|---|---|---|
| loading | Treatment | total | weight (gram)/L | after |
| COD | hours | volume (L) | wastewater | treatment |
| 520 | 6 | 6 | 60 | 147 |
| 460 | 8 | 6 | 60 | 99 |
| 480 | 10 | 6 | 60 | 84 |
| 475 | 12 | 6 | 60 | 66 |

Attachment of the activated sludge to the polymer paper carriers was also studied in the cultivation containers. The sludge attachment status and clarity of supernatant were observed. Table 21 shows the results, indicating clear supernatant and almost complete attachment of the activated sludge while mixed either by air-sparging or the rotating wheels.

TABLE 21

| Oxygen transfer | Polymer paper | Sludge attachment | Wastewater |
|---|---|---|---|
| method | carriers | status | clear? |
| Air-sparging | With | Attached | Yes |
| Air-sparging | Without | In suspension | No |
| Rotating wheel | With | Attached | Yes |
| Rotating wheel | Without | In suspension | No |

In order to understand attachment and growth of the activated sludge and related microorganisms in detail, scanning electronic microscope was used. Our results showed clear microorganism growth and attachment on and within the carriers.

### Example 22

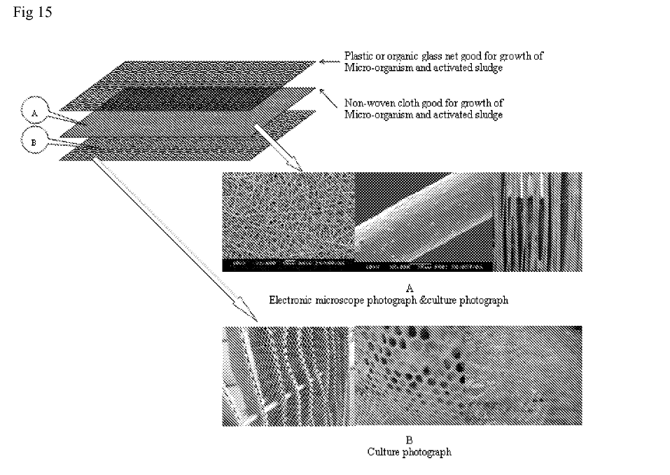

We observed that micro-organisms grew within non-woven polymer fibers as well as on plastic and organic glass net (FIG. 15). We then examined non-woven polymer fibers, plastic net, organic glass net, aluminum net, aluminum alloy net and stainless net for attached cultivating activated sludge and microorganism. We discovered that non-woven polymer fiber sheet and nets made of plastic, organic glass better for attached growth of the activated sludge and microorganism than that of aluminum, aluminum alloy and stainless (FIG. 15). Thus, we used non-woven polymer fiber packed between 1-2 layers of plastic or organic glass nets as a carrier for attached growth of the activated sludge and microorganism (FIG. 15). Through 6 month experiments, we have observed no erosion occurred on the surface of these materials.

### Example 23

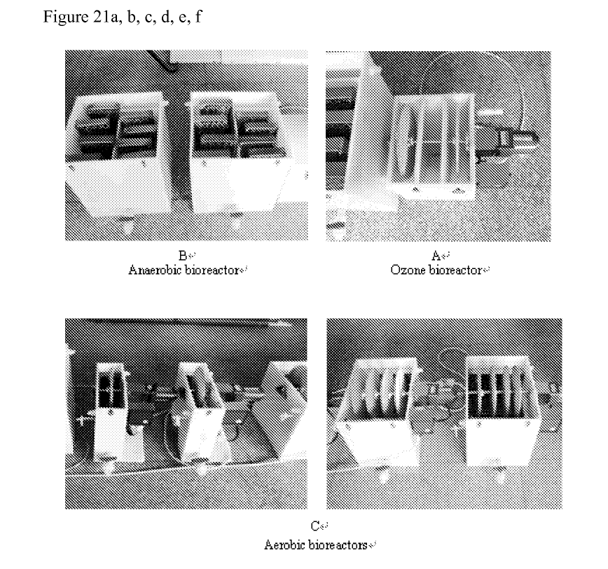

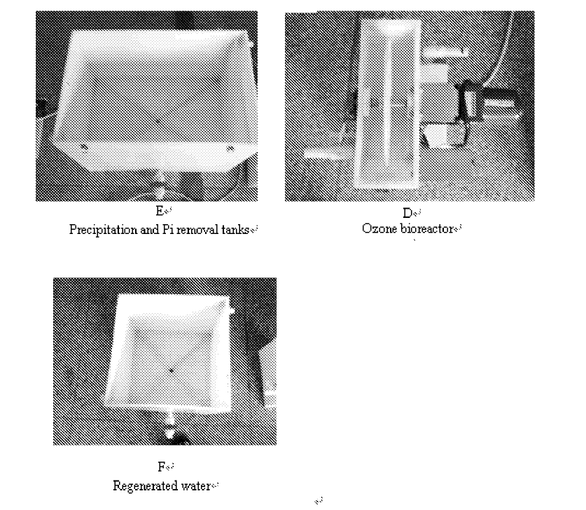

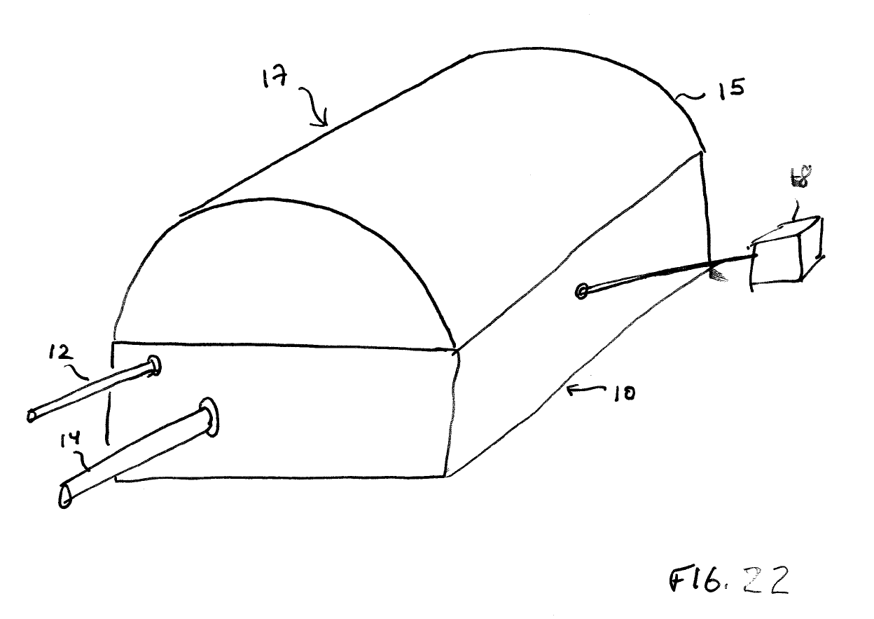

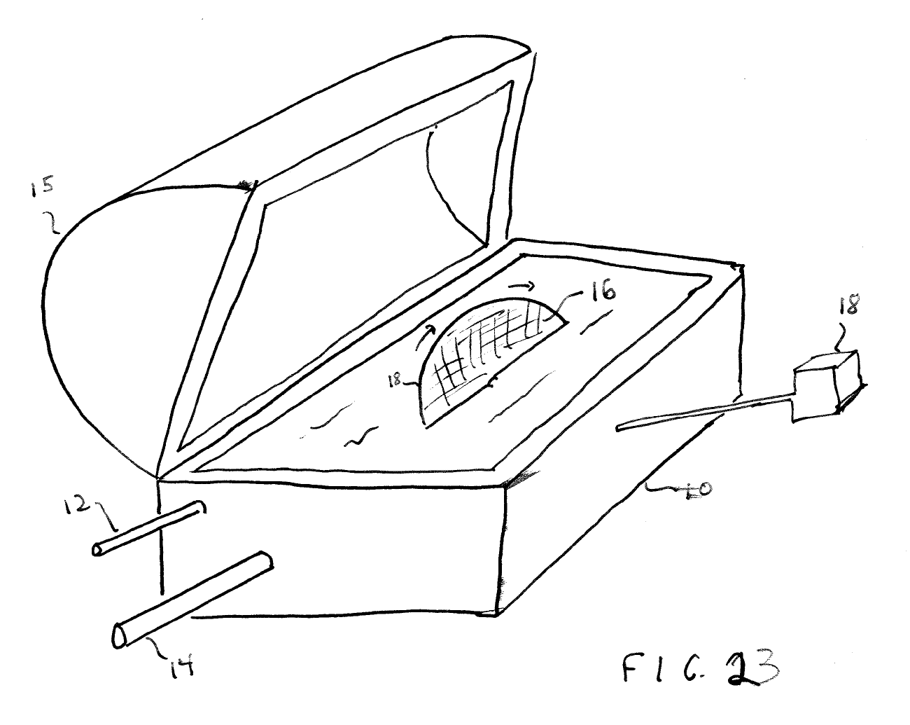

A perfusion system using a 0.25 meter-diameter rotating wheel cultivation system was used for process development. The prototype system was shown in FIGS. 21a-f. The complete system includes anaerobic fermentation, aerobic fermentation, O₃ treatment and Pi removal. This system was used to study wastewater treatment. The treatment results of 4 hours retention time for aerobic fermentation were shown in Table 22. In addition, a fish tank was used to biologically monitor the water quality. No fish death was observed in a week. Surprisingly, total phosphate (TP) and total ammonia nitrogen (NH4-N) were significantly reduced when fishes were raised together with water plants. For example, TP was reduced from 0.8 to 0.2 mg/L while NH4-N was reduced from 20.0 to 7.1 mg/L. These results indicated use of water plants together with fishes in the final clear water tank to reduce TP and NH4-N.

TABLE 22

| | | | | | One week |
|---|---|---|---|---|---|
| Loading | Retention | COD after | Color after | Odor after | fish raising |
| COD | time | treatment | treatment | treatment | fish in a tank |
| 1047 | 7 | 25 | No | No | alive |
| 240 | 4 | 8 | No | No | alive |
| 308 | 4 | 12 | No | No | alive |
| 157 | 4 | 9 | No | No | alive |

After several runs, it was concluded that the loading richness of the wastewater such as COD levels automatically regulate biomass of the activated sludge. For example, higher COD loading of the wastewater resulted in higher biomass of the activated sludge attached to the paper carrier. The lower COD loading of the wastewater resulted in lower biomass of the activated sludge. This feature favors very much a stable perfusion cultivation system for wastewater treatment.

Very surprisingly, we used O₃ treatment before aerobic fermentation unit, our results indicated that O₃ treatment at 10 mg/L level reduced COD from 1001 to 650, and reduced ammonia nitrogen from 37 to 19 mg/L. These surprising results indicated that combined use of O₃ unit and aerobic fermentation unit added one more choice for process development of wastewater treatment.

### Example 24

A perfusion system using a 1.0 meter-diameter rotating wheel cultivation system was designed for a 120-ton daily treatment facility. The complete system includes anaerobic fermentation, aerobic fermentation, O₃ treatment and Pi removal. This system was used to study 120 tons/day wastewater treatment. COD removal 200-500 gram/ton/hour for each aerobic unit (2 ton work volume per unit) were expected. Table 23 shows the results of 4 hours of perfusion cultivation retention time at 25-30° C. plus two hour ozone treatment. A fish tank was again used to biologically monitor the outlet water quality. We concluded that a stable wastewater treatment system has been established.

Again, we found that total phosphate (TP) and total ammonia nitrogen (NH4-N) were significantly reduced when fishes were raised together with water plants. For example, TP was reduced from 1.2 to 0.4 mg/L while NH4-N was reduced from 16.0 to 4.6 mg/L. These results again indicated use of water plants together with fishes in the final clear water tank to reduce TP and NH4-N.

TABLE 23

| | | | | | | One |
|---|---|---|---|---|---|---|
| Loading | Reten- | | | | | week |
| waste- | tion | COD | | Color | Odor | raising |
| water | time | after | Total | after | after | fish in |
| COD | (hour) | treatment | Phosphate | treatment | treatment | a tank |
| 690 | 4 | 43 | 0.3 mg/L | No | No | Alive |
| 598 | 4 | 36 | 0.2 mg/L | No | No | Alive |
| 459 | 4 | 26 | 0.2 mg/L | No | No | Alive |
| 356 | 4 | 24 | 0.1 mg/L | No | No | Alive |
| 278 | 4 | 16 | 0.1 mg/L | No | No | Alive |
| 182 | 4 | 8 | 0.1 mg/L | No | No | Alive |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A rotating wheel assembly for introducing an ambient gas into a liquid, comprising a wheel plate having a face; and a net structure on the face.

2. The rotating wheel assembly of claim 1, further comprising an axle passing through the wheel plate, the axle being positioned such that a portion of the wheel plate protrudes above the level of the liquid.

3. The rotating wheel assembly of claim 1, wherein the net structure is made of aluminum, aluminum alloy, stainless steel, or ozone-resistant plastic.

4. The rotating wheel assembly of claim 1, wherein the net structure comprises one or more layers of plastic or metal net on the face.

5. The rotating wheel assembly of claim 4, wherein the net has mesh having a shape of diamond, square, or hexagon.

6. The rotating wheel assembly of claim 4, wherein the net has mesh of 0.5-2.0 cm in diameter.

7. The rotating wheel assembly of claim 4, wherein a wheelward-most layer from the layers of net comprises a plurality of meshes having a first size, and a wheelward-least layer from the plurality of net layers comprises a plurality of net meshes having a second size, wherein the second size is equal to or larger than the first size.

8. The rotating wheel assembly of claim 1, wherein the face is a side face or a circumferential face of the wheel plate.

9. (canceled)

10. The rotating wheel assembly of claim 1, wherein the wheel assembly comprises a plurality of wheel plates, each having a face for placement of the net structure.

11. The rotating wheel assembly of claim 10, wherein the wheel assembly comprises:
a first wheel plate;
a second wheel plate mounted coaxially with the first wheel plate and separated from the first wheel plate along an axial direction; and
a plurality of boards extending between the first and second wheels, the boards having faces;
wherein the net structure is disposed on the faces of the boards.

12. The rotating wheel assembly of claim 1, wherein the wheel is a rolling tube covered by one or more layers of the net structures.

13. (canceled)

14. (canceled)

15. A bioreactor for introducing an ambient gas into a liquid, comprising:
a tank for holding the liquid; and
a wheel assembly of claim 1 rotatably mounted in the tank.

16. The bioreactor of claim 15, further comprising an airtight lid for the tank.

17. The bioreactor of claim 15, wherein the gas is air, oxygen, ozone, fragrant gas, N₂, or CO₂.

18. The bioreactor of claim 15, wherein the net structure is made of plastic, aluminum, aluminum alloy, stainless steel and the gas is air, oxygen, fragrant gas, N₂, or CO₂; or the net structure is made of ozone-resistant plastic and the gas is ozone.

19. (canceled)

20. (canceled)

21. The bioreactor of claim 16, wherein the liquid contains chemicals, virus, microorganisms, plant cells, or mammalian cells.

22. The bioreactor of claim 21, wherein the microorganisms are bacteria or yeast.

23. The bioreactor of claim 15, wherein the liquid contains water, industry wastewater, or sewage.

24. A method of liquid treatment, the method comprising repeatedly moving a net structure through a gas to be dissolved in the liquid; and plunging the net structure into the liquid, wherein the liquid is in a tank.

25. The method of claim 24, wherein the gas is air, oxygen, ozone, fragrant gas, N₂, or CO₂.

26. The method of claim 24, wherein the net structure is made of aluminum, aluminum alloy, stainless steel, or plastic, and the gas is air, oxygen, fragrant gas, N₂, or CO₂; or the net structure is made of ozone-resistant plastic and the gas is ozone.

27. (canceled)

28. The method of claim 24, wherein the tank is airtight.

29. The method of claim 28, wherein the liquid contains chemicals, virus, microorganisms, plant cells, or mammalian cells; or the liquid contains water, industry wastewater or sewage, culture medium, or broth.

30. The method of claim 29, wherein the microorganisms are bacteria or yeast.

31. (canceled)

32. (canceled)

33. The bioreactor of claim 15, wherein the tank further comprises activated sludge.

34. The bioreactor of claim 15, wherein the activated sludge is grown on a matrix.

35. The bioreactor of claim 34, wherein the matrix contains a layer of polymer non-woven cloth that is in between plastic nets.
